(19) United States
(12) Patent Application Publication
(10) Pub. No.: US 2001/0018427 A1
(43) Pub. Date: Aug. 30, 2001

(54) Method of using a scavenger receptor in the treatment of atherosclerosis

(75) Inventors: Charles Larry Bisgaier, Ann Arbor, MI (US); Joseph Anthony Cornicelli, Ann Arbor, MI (US); Sabine Woelle, Ann Arbor, MI (US)

Correspondence Address: MERCHANT & GOULD, P O BOX 2903, MINNEAPOLIS, MN, 55402-0903, US

(73) Assignee: Warner-Lambert Company, Ann Arbor, MI (US)

(21) Appl. No.: 09/836,733

(22) Filed: Apr. 17, 2001

Related U.S. Application Data
(63) Continuation of application No. 09/264,195, filed on Mar. 8, 1999.
Application No. 09/264,195 is a continuation of application No. 08/897,163, filed on Jul. 18, 1997, now abandoned.
Application No. 08/897,163 is a continuation of application No. 08/319,183, filed on Oct. 6, 1994, now abandoned.

(51) Int. Cl. A61K 48/00

(52) U.S. Cl. 514/44; 435/320.1

(57) ABSTRACT

A method for introducing a scavenger receptor gene into a mammal to make said mammal resistant to atherosclerosis; an artificial scavenger receptor minigene or partial minigene as well as the ectopic expression of a scavenger receptor in the liver of a mammal for the reduction of apo B containing lipoproteins, elevation of high-density lipoprotein cholesterol, and prevention of atherosclerosis; and a method of treating atherosclerosis, hyperbetalipoproteinemia (i.e., high-levels of apolipoprotein (apo) B containing lipoproteins), hypercholesterolemia, hypertriglyceridemia; hypoalphalipoproteinemia (i.e., low levels of high-density lipoprotein), vascular complications of diabetes, transplant, atherectomy, and angioplastic restenosis in a patient with a therapeutically effective amount of a scavenger receptor gene alone or combined with a ACAT inhibitor, a HMG-CoA reductase inhibitor, a bile acid sequestrant, or lipid regulator, and pharmaceutical delivery methods which include these agents.

### BACKGROUND OF THE INVENTION

The present invention relates to a medical method of treatment. In particular, the present invention concerns the use of a scavenger receptor gene (SR) to make a mammal resistant to atherosclerosis, to methods for their production, to pharmaceutical delivery methods which include these genes, and to pharmaceutical methods of treatment. In particular, the novel SR gene is useful in treating hyperbetalipoproteinemia (i.e., high levels of apolipoprotein (apo) B containing lipoproteins), hypercholesterolemia, hypertriglyceridemia, hypoalphalipoproteinemia (i.e., low levels of high-density lipoprotein cholesterol), vascular complications of diabetes, transplant, atherectomy, and angioplastic restenosis. More particularly, the novel SR gene alone or combined with another agent for the treatment of atherosclerosis such as, for example, an ACAT inhibitor, a HMG-CoA reductase inhibitor, a lipid regulator, a bile acid sequestrant, and the like is useful in the treatment of atherosclerosis.

The macrophage is thought to play a pivotal role in the pathogenesis of atherosclerosis (Brown M. S., Goldstein J. L., Krieger M., Ho Y. K., Anderson R. G. W., J. Cell Biol., 82:597-613 (1979); Goldstein J. L., Ho Y. K., Basu S. K., Brown M. S., Proc. Natl. Acad. Sci. USA, 76:333-337 (1979); Brown M. S., Goldstein J. L., Ann. Review Biochem., 52:223-261 (1983); Steinberg D., Parthasarathy S., Carew T. E., Khoo J. C., Witztum, J. L., N. Engl. J. Med., 320:915-924 (1989); Carew T. E., Am. J. Cardiol., 64:18G-22G (1989); Brown M. S., Goldstein J. L., Nature, 343:508-509 (1990); Kurihara Y. A., Matsumoto A., Itakura H., Kodama T., Current Opinion in Lipidology, 2:295-300 (1991); Krieger M., TIBS, 17:141-146 (1992)). SRs are present on macrophages and mediate binding and internalization of a broad variety of ligands including modified apo B containing lipoproteins (Brown M. S., Goldstein J. L., Krieger M., Ho Y. K., Anderson R. G. W., supra., 1979; Goldstein J. L., Ho Y. K., Basu S. K., Brown M. S., supra., 1979; Brown M. S., Goldstein J. L., supra., 1983; Steinberg D., Parthasarathy S., Carew T. E., Khoo J. C., Witztum J. L., supra., 1989; Carew T. E., supra., 1989; Brown M. S., Goldstein J. L., supra., 1990; Kurihara Y. A., Matsumoto A., Itakura H., Kodama T., supra., 1991; Krieger M., supra., 1992; Fogelman A. M., Shechter I., Seager J., Hokom M., Child J. S., Edwards P. A., Proc. Natl. Acad. Sci. USA, 77:2214-2218 (1980); Haberland M. E., Fogelman A. M., Proc. Natl. Acac. Sci. USA, 82:2693-2697 (1985); Horiuchi S., Murakami M., Takata K., Morino Y., J. Biol. Chem., 261:4962-4966 (1986); Dresel H. A., Friedrich E., Via D. P., Sinn H., Ziegler R., Schettler G., EMBO Journal, 6:319-326 (1987); Quinn M. T., Parthasarathy S., Fong L. G., Steinberg D., Proc. Natl. Acad. Sci. USA, 84:2995-2998 (1987); Takata K., Horiuchi S., Araki N., Shiga M., Saitoh M., Morino Y., J. Biol. Chem., 263:14819-14825 (1988); Wright T. L., Roll F. J., Jones A. L., Weisiger R. A., Gastroenterology, 94:442-452 (1988); Takata K., Horiuchi S., Morino Y., Biochim. Biophys. Acta., 984:273-280 (1989); Eskild W., Kindberg G. M., Smedsrød B., Blomhoff R., Norum K. R., Berg T., Biochem. J., 258:511-520 (1989); Steinbrecher U. P., Lougheed M., Kwan W. -C., Dirks M., J. Biol. Chem., 264:15216-15223 (1989); Hampton R. Y., Golenbock D. T., Penman M., Krieger M., Raetz C. R. H., Nature, 352:342-344 (1991); Stehle G., Friedrich E. A., Sinn H., et al., J. Clin. Invest., 90:2110-2116 (1992); Tokuda H., Masuda S., Takakura Y., Sezaki H., Hashida M., Biochem. Biophys. Res. Commun., 196:18-24 (1993); de Vries H. E., Kuiper J., de Boer A. G., van Berkel T. J. C., Breimer D. D., J. Neurochem., 61:1813-1821 (1993); Pearson A. M., Rich A., Krieger M., J. Biol. Chem., 268:3546-3554 (1993); Zhang H., Yang Y., Steinbrecher U. P., J. Biol. Chem., 268:5535-5542 (1993); Dunne D. W., Resnick D., Greenberg J., Krieger M., Joiner K. A., Proc. Natl. Acad. Sci. USA, 91:1863-1867 (1994); Freeman M. W., Current Opinion in Lipidology, 5:143-148 (1994)). The SR may also be present on smooth muscle and endothelial cells under specific circumstances (Bickel P. E., Freeman M. W., J. Clin. Invest., 90:1450-1457 (1992)). Unlike the low-density lipoprotein (LDL) receptor, the SR lacks negative feedback regulation by cholesterol allowing the sustained uptake of modified lipoprotein and transformation of macrophages into foam cells (Brown M. S., Goldstein J. L., Krieger M., Ho Y. K., Anderson R. G. W., supra., 1979; Goldstein J. L., Ho Y. K., Basu S. K., Brown M. S., supra., 1979; Brown M. S., Goldstein J. L., supra., 1983). The macrophage derived foam cell is characteristic of early atherosclerotic lesions in a variety of species including humans; its accelerated formation can be mimicked in a variety of animal models fed cholesterol-enriched diets (Mahley R. W., Athero. Rev., 5:1-34 (1979); Clarkson T. B., Shively C. A., Weingand K. W., Comp. Anim. Nutr., 6:56-82 (1988)).

Two forms of bovine SRs, Type I and II, have been cloned from bovine lung libraries (Kodama T., Freeman M., Rohrer L., Zabrecky J., Matsudaira P., Kreiger M., Nature, 343:531-535 (1990); Rohrer L., Freeman M., Kodama T., Penman M., Kreiger M., Nature, 343:570-572 (1990)). These trimeric structurally similar receptors are derived from alternate splicing of a single gene product resulting in SR that contain (Type I) or lack (Type II) the carboxyl terminal cysteine-rich domain (Freeman M., Ashkenas J., Rees D. J. G., et al., Proc. Natl. Acad. Sci. USA, 87:8810-8814 (1990)). Although the cysteine-rich domain (i.e., Type I, Domain VI) is highly conserved between species, its functional significance is not known. Mutagenesis studies of Acton, et al. (Acton S., Resnick D., Freeman M., Ekkel Y., Ashkenas J., Krieger M., J. Biol. Chem., 268:3530-3537 (1993)), suggest the collagenous domains (Domain V) present in both Type I and II SR contain the sequence necessary for recognition of polyanionic ligands. Structural studies of Penman, et al. (Penman M., Lux A., Freedman N. J., et al., J. Biol. Chem., 266:23985-23993 (1991)), have suggested that the assembly of SR into trimers involves the noncovalent association of a spacer domain (i.e., Domain III) disulfide linked dimer with a monomer. The trimeric SR structure, however, does not appear to be requisite for functional binding since monomers are fully capable of binding ligands (Via D. P., Kempner E. S., Pons L., et al., Proc. Natl. Acad. Sci. USA, 89:6780-6784 (1992)).

Although overwhelming circumstantial evidence suggest modified LDL exists in vivo (Carew T. E., supra., 1989), their presence has been viewed with skepticism since these particles have not been isolated from plasma. It is likely their compartmentalized formation in the subendothelium and rapid uptake by resident macrophages prevent any accumulation in plasma. However, in vitro and in vivo, macrophage SR avidly bind, internalize, and-degrade chemically modified LDL. Although smooth muscle cell and macrophage SR expression in the artery wall may play a role in lesion formation, their presence in liver may portend a protective role (Brown M.S., Goldstein J. L., supra. 1990). Indeed, nonparenchymal liver cells, including Kupffer and endothelial cells are capable of binding and degradation of acetylated and oxidized LDL (Dresel H. A., Friedrich E., Via D. P., Sinn H., Ziegler R., Schettler G., supra., 1987; van Berkel T. J. C., Nagelkerke J. F., Kruijt J. K., FEBS Letters, 132:61-66 (1981); Dresel H. A., Friedrich E., Via D. P., Schettler G., Sinn H., EMBO Journal, 4:1157-1162 (1985); de Rijke Y. B., van Berkel T. J. C., J. Biol. Chem., 269:824-827 (1994)). Furthermore, intravenously infused acetylated LDL accumulates primarily in hepatic sinusoidal and endothelial cells, and to a lesser extent in Kupffer cells (Dresel H. A., Friedrich E., Via D. P., Sinn H., Ziegler R., Schettler G., supra., 1987; Dresel H. A., Friedrich E., Via D. P., Schettler G., Sinn H., supra., 1985; Nagelkerke J. F., Barto K. P., van Berkel T. J. C., J. Biol. Chem., 258:12221-12227 (1983); Pitas R. E., Boyles J., Mahley R. W., Montgomery B. D., J. Cell Biol., 100:103-117 (1985); Horiuchi S., Takata K., Maeda H., Morino Y., J. Biol. Chem., 259:53-56 (1985); van Berkel T. J. C., de Rijke Y. B., Kruijt J. K., J. Biol. Chem., 266:2282-2289 (1991)). Studies utilizing oxidized LDL have instead primarily demonstrated ligand accumulation in Kupffer, and to a lesser extent in endothelial cells (Esbach S., Pieters M. N., Van der Boom J., et al., Hepatology, 18:537-545 (1993)). Acetylated LDL uptake by hepatic parenchyma occurs at a near negligible rate (Nagelkerke J. F., Barto K. P., van Berkel T. J. C., supra., 1983). Overall, these studies suggest the liver nonparenchymal cells have the capacity to remove potentially atherogenic lipoproteins.

Thus, an object of the present invention is the ectopic expression of a SR in mammalian cells, and in particular hepatic cells that do not normally express it. It has surprisingly and unexpectedly been found that expression of the SR in liver cells caused a drop in apo B containing lipoprotein and an elevation in high-density lipoprotein (HDL) and a favorable change in the ratio of apo B containing lipoprotein cholesterol to HDL cholesterol. Further, it has unexpectedly been found that liver, containing these ecotopically expressed SRs, is protected from cholesterol accumulation and does not store excess lipids.

### SUMMARY OF THE INVENTION

Accordingly, the present invention is directed to a method for introducing a SR gene into the liver of a mammal to make said mammal resistant to atherosclerosis, comprising introducing the DNA into a mammal by a process of delivery selected from the group consisting of:

(a) use of calcium phosphate coprecipitation;

(b) in a complex of cationic liposomes;

(c) electroporation;

(d) receptor-mediated endocytosis;

(e) naked DNA;

(f) transduction by a viral vector;

(g) particle-mediated gene transfer; and

(h) synthetic peptides.

In a preferred embodiment of the first aspect of the invention, the mammal is a human.

In a second aspect, the present invention is directed to a method for introducing a SR gene into a mammal to make said mammal resistant to atherosclerosis comprising inserting said SR gene into a vector and expressing the SR in the liver of said mammal.

In a preferred embodiment of the second aspect of the invention, the mammal is a human.

In a third aspect, the present invention is directed to an artificial SR minigene or partial minigene comprising:

(a) a liver specific promoter or wherein the liver specific promotor is absent;

(b) a 5′ untranslated region or wherein the 5′ untranslated region is absent;

(c) a coding sequence;

(d) a 3′ untranslated region or wherein the 3′ untranslated region is absent; and

(e) a polyadenylation signal or wherein the polyadenylation signal is absent.

In a preferred embodiment of the third aspect of the invention the 5′ untranslated region is selected from the group consisting of: a 5′ untranslated region containing natural (heterologous or homologous) nucleotides; a 5′ untranslated region containing synthetic nucleotides; and a 5′ untranslated region containing a combination of natural (heterologous or homologous) and synthetic nucleotides.

In a more preferred embodiment of the third aspect of the invention the 5′ untranslated region is selected from a group consisting of: a 5′ untranslated region between the promoter and translation initiation site(s) of the SR coding region; and a 5′ untranslated region excluding a 5′ untranslated region between the promoter and translation initiation site(s) of the SR coding region.

In a most preferred embodiment of the third aspect of the invention the 5′ untranslated region between the promotor and the SR coding region is 5 bp of the 5′ untranslated region of the SR.

In a preferred embodiment of the third aspect of the invention the 3′ untranslated region is selected from the group consisting of: a region between the 3′ end of the SR coding sequence and the 3′ end of a sequence containing a poly-A tail consisting of natural (heterologous or homologous) nucleotides; a region between the 3′ end of the SR coding sequence and the 3′ end of a sequence containing a poly-A tail consisting of synthetic nucleotides; and a region between the 3′ end of the SR coding sequence and the 3′ end of a sequence containing a poly-A tail consisting of a combination of natural (heterologous or homologous) and synthetic nucleotides.

In a more preferred embodiment of the third aspect of the invention the 3′ untranslated region is selected from the group consisting of: a region between the 3′ end of the SR coding sequence and a 5′ end of a sequence containing a poly-A tail; and exclusion of a region between the 3′ end of the SR coding sequence and a 5′ end of a sequence containing a poly-A tail.

In a most preferred embodiment of the third aspect of the invention the 3′ untranslated region is truncated at the specific restriction site using the enzyme Asp700 or any other isochizomer of Asp700.

In a preferred embodiment of the third aspect of the invention the polyadenylation signal is selected from the group consisting of: a polyadenylation signal containing natural (heterologous or homologous) nucleotides; a polyadenylation signal containing synthetic nucleotides; and a polyadenylation signal containing a combination of natural (heterologous or homologous) and synthetic nucleotides.

In a more preferred embodiment of the third aspect of the invention, the polyadenylation signal is the human growth hormone sequence spanning the polyadenylation signal.

In a most preferred embodiment of the third aspect of the invention the polyadenylation signal is 650 bp sequence of the human growth hormone sequence spanning the polyadenylation signal.

In a preferred embodiment of the third aspect of the invention the liver specific promoter is the mouse transferrin promoter.

In a more preferred embodiment of the third aspect of the invention the coding sequence is selected from the group consisting of: the complete coding sequence; a truncated form of the coding sequence; and fragments of the complete coding sequence including insertions, deletions, and repetitions.

In a fourth aspect, the present invention is directed to the ectopic expression of a SR in the liver of a mammal for the reduction of apo B containing lipoproteins, elevation of high-density lipoprotein cholesterol, and prevention of atherosclerosis.

In a preferred embodiment of the fourth aspect of the invention, the mammal is a human.

In a more preferred embodiment of the fourth aspect of the invention the expression is transient expression in the liver.

In a most preferred embodiment of the fourth aspect of the invention the expression is stable expression in the liver.

In a fifth aspect, the present invention is directed to a method of treating atherosclerosis; hyperbetalipoproteinemia; hypercholesterolemia; hypertriglyceridemia; hypoalphalipoproteinemia; vascular complications of diabetes; transplant, atherectomy, and angioplastic restenosis (Groves P. H., Lewis M. J., Cheadle H. A., Penny W. J., Circulation, 87:590-597 (1993); More R. S., Rutty G., Underwood M. J., Gershlick A. H., J. Pathol., 172:287-292 (1994)) in a patient comprising administering to the liver of said patient a therapeutically effective amount of a SR gene.

In a sixth aspect, the present invention is directed to a method of treating atherosclerosis; hyperbetalipoproteinemia; hypercholesterolemia; hypertriglyceridemia; hypoalphalipoproteinemia; vascular complications of diabetes; transplant, atherectomy, and angioplastic restenosis in a patient comprising administering to the liver of said patient a therapeutically effective amount of a SR gene in combination with one or more agents selected from the group consisting of:

(a) ACAT inhibitor;

(b) HMG CoA reductase inhibitor;

(c) lipid regulator; and

(d) bile acid sequestrant.

In a seventh aspect, the present invention is directed to a pharmaceutical delivery method adapted for hepatic administration to a patient in an effective amount of an agent for treating atherosclerosis; hyperbetalipoproteinemia; hypercholesterolemia; hypertriglyceridemia; hypoalphalipoproteinemia; vascular complications of diabetes; transplant, atherectomy, and angioplastic restenosis comprising a SR gene and a suitable viral or nonviral delivery system.

In a preferred embodiment of the seventh aspect of the invention, the pharmaceutical delivery method is adapted for ex vivo or in vivo delivery.

In a most preferred embodiment of the seventh aspect of the invention, the pharmaceutical delivery method is directed to therapeutic or prophylactic administration.

### BRIEF DESCRIPTION OF THE DRAWINGS

The invention is further described by the following nonlimiting examples which refer to the accompanying FIGS. 1 to 10, short particulars of which are given below.

FIG. 1

The 5.2 kb construct of the bovine SR Type I minigene. A full length bovine SR cDNA (black bar) was truncated in the 3′ untranslated region at the single restriction site Asp700. The ˜1.6 kb fragment was then ligated to a 0.65 kb containing the poly A signal sequence of the human growth hormone gene (white bar) using the Sma I and Asp700 fusion site. At the 5′ end, a 3 kb DNA fragment of the mouse transferrin promoter (stippled box) was attached using Bam HI. The minigene was inserted into a pGem 11Zf (−) using Eco RI at the 5′ end and Not I at the 3′ end. The region between the Bam HI site at the 3′ end of the mouse transferrin promoter and the first ATG codon of the SR cDNA contained 5 base pairs (5′-gaagt-3′) of the untranslated region of the bovine SR allowing the first ATG to be in the optimal context for translation initiation (Kozak M., Cell, 47:481-483 (1986); Kozak M., J. Cell Biol., 108:229-241 (1989)).

FIG. 2

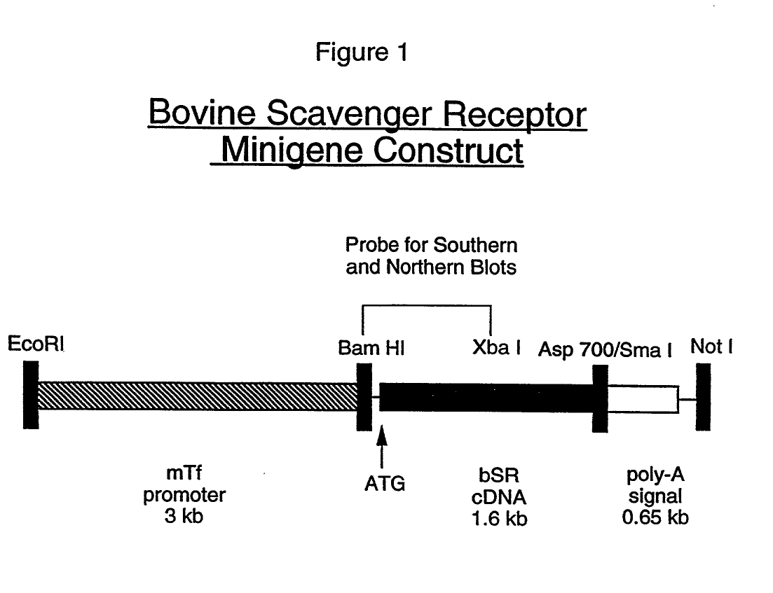

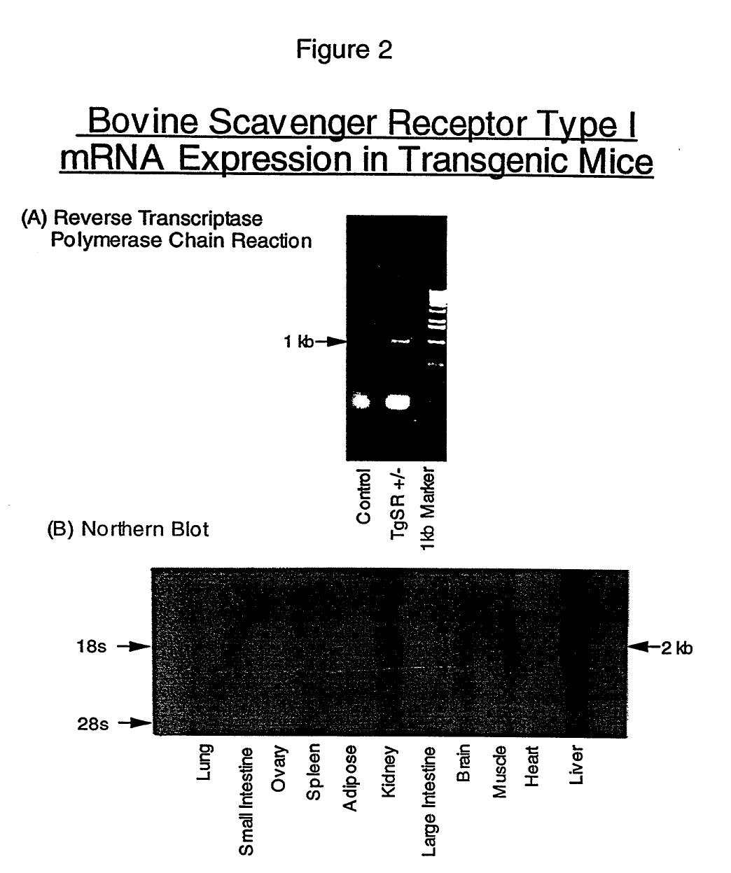

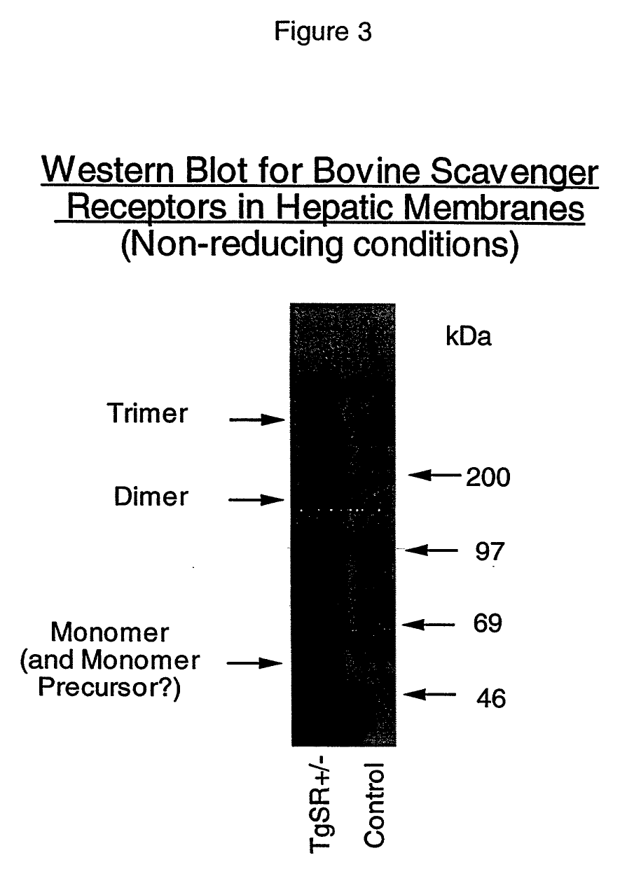

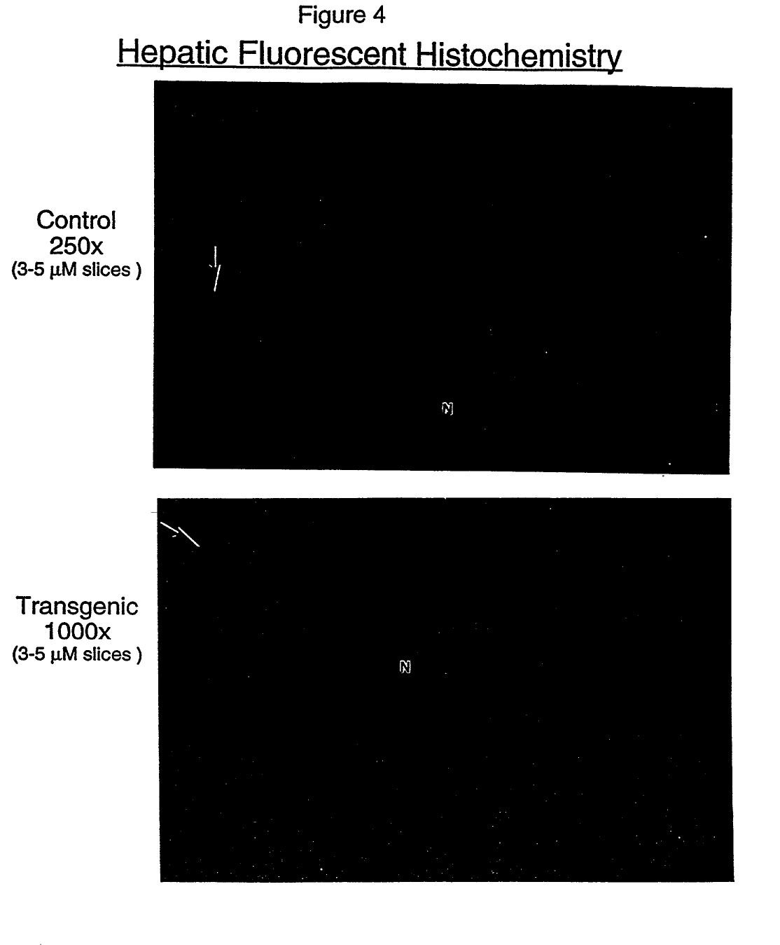

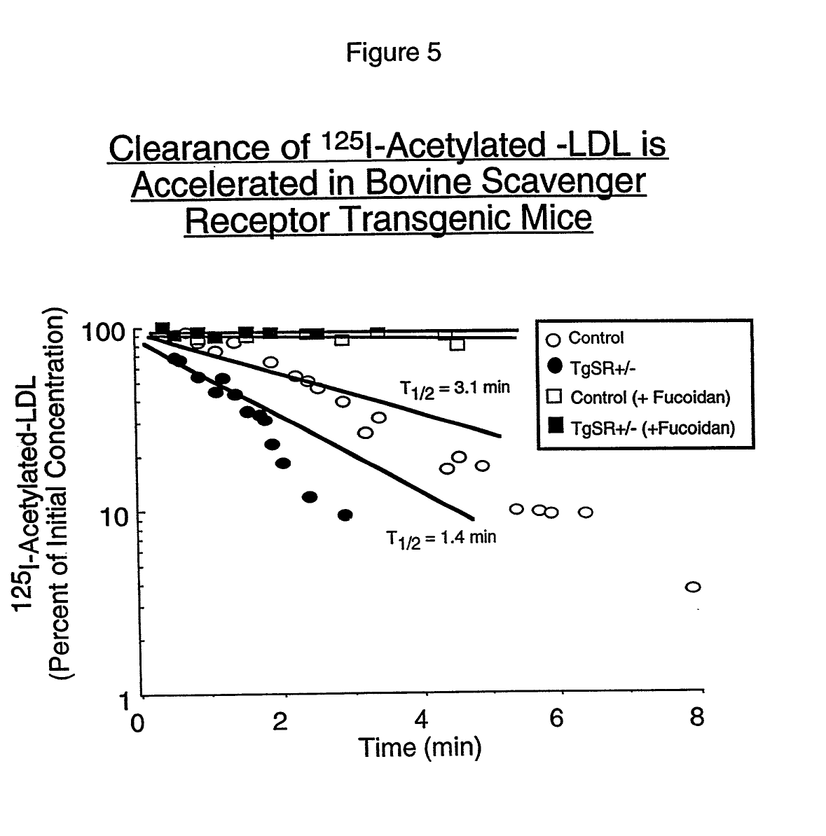

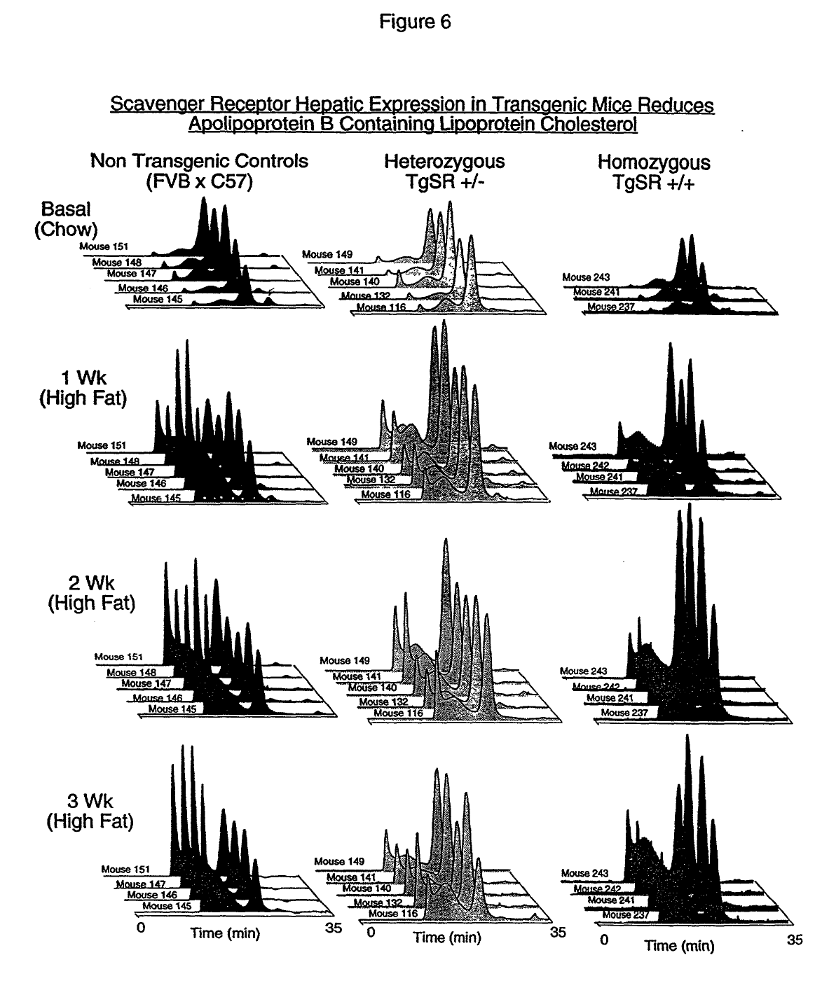

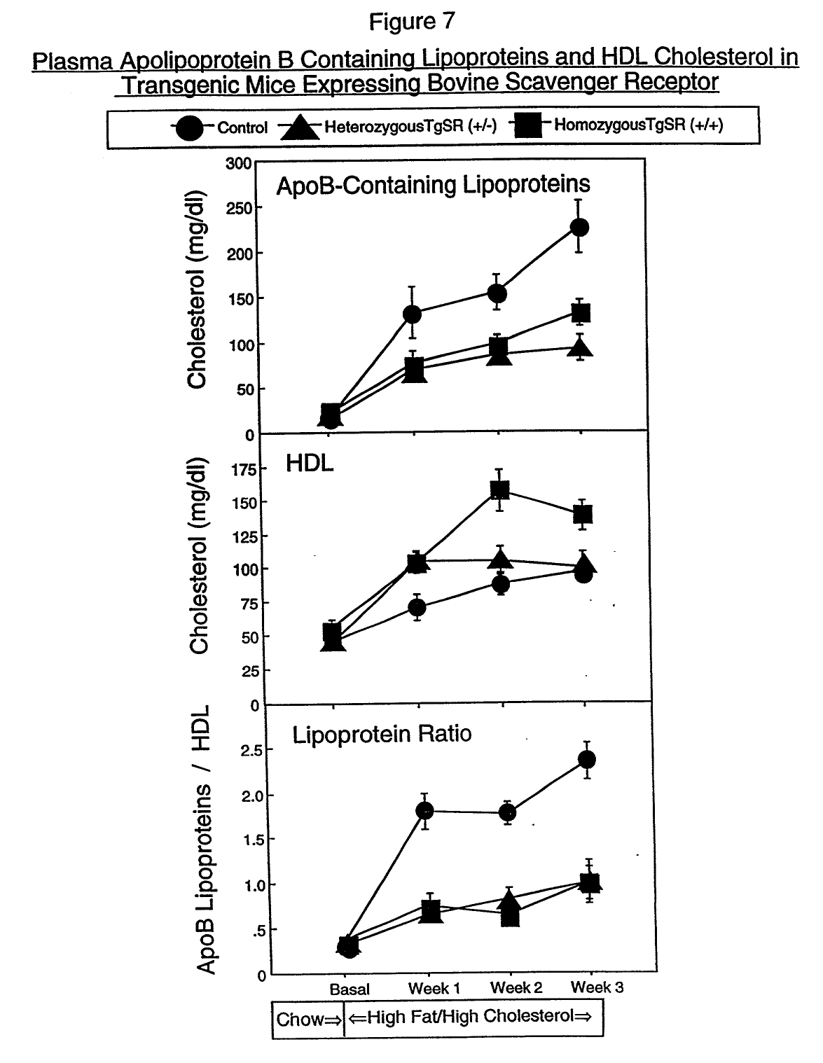

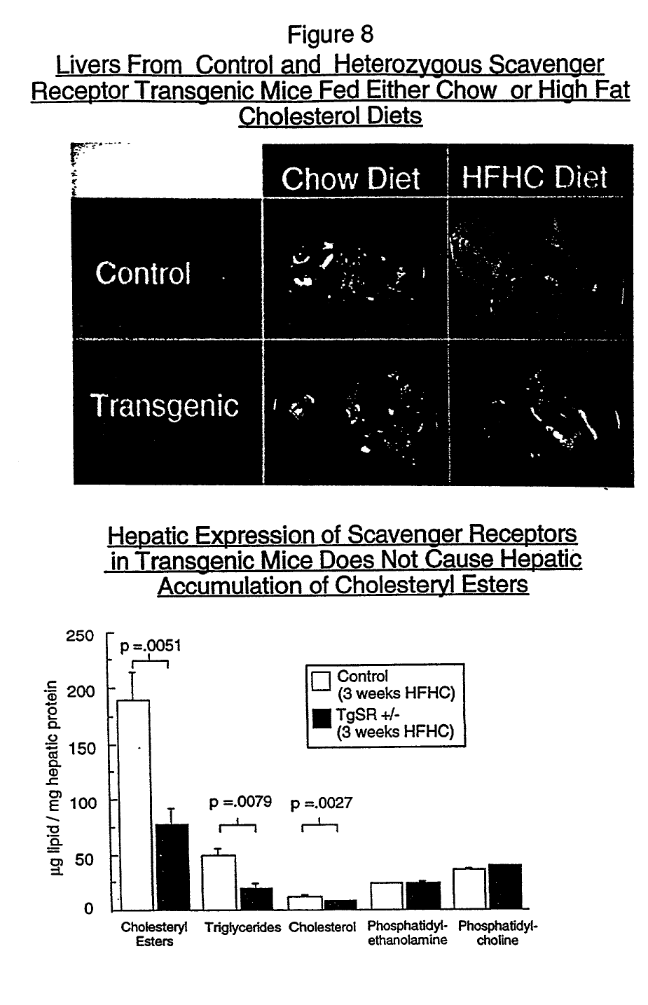

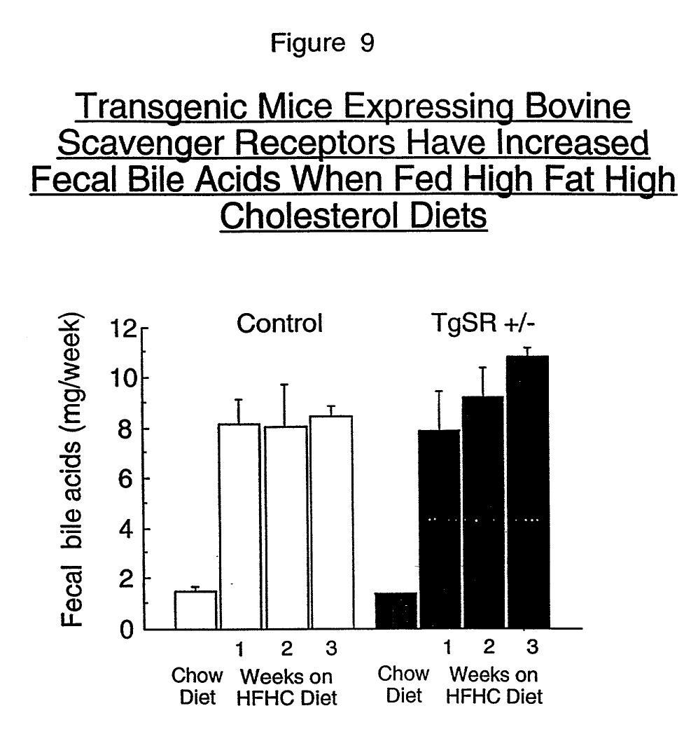

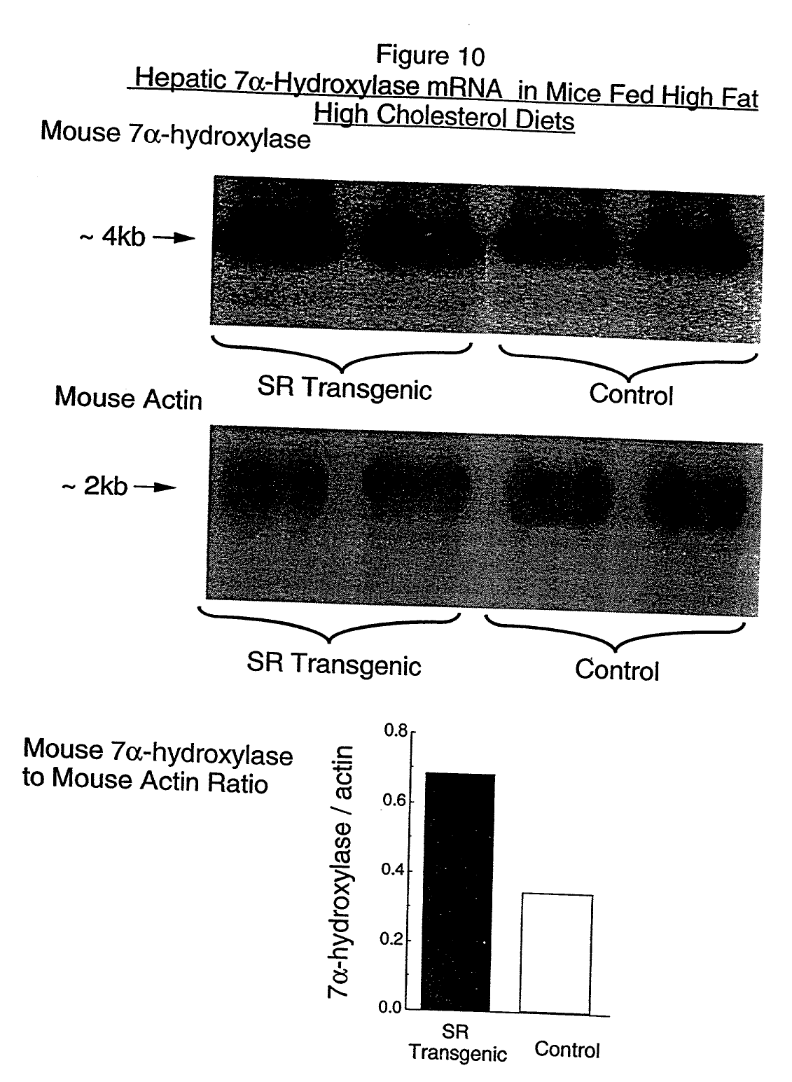

(A) Reverse transcriptase-polymerase chain reaction of hepatic RNA from a control and a TgSR+/− mouse shows the presence of a 1 kb amplified region of bovine SR mRNA. Reference DNA size standards are shown (1 kb marker). (B) Northern blot analysis of a bovine SR Type I expressing mouse. For each tissue sample 10 μg of total RNA was electrophoretically fractionated on a formaldehyde-1% agarose gel, transferred onto Zetaprobe membranes, and then hybridized to a bovine SR Type I specific cDNA probe as indicated in FIG. 1. The probe did not hybridize to RNA isolated from control mouse tissues under the same conditions (not shown). The blot was washed and exposed to X-Omat AR film at −80° C. overnight.

FIG. 3

Western blot analysis of a TgSR+/− and control mouse liver membrane preparation. Nonreduced membrane protein (22.5 μg/lane) was loaded onto 7.5% SDS polyacrylamide gel and transferred to nitrocellulose membranes and the presence of bovine SR were determined as described in Example 5. Monomeric plus possibly monomeric precursors, dimeric, and trimeric forms of the bovine SR are apparent.

FIG. 4

Hepatic fluorescent histochemistry following DiI-acetylated human LDL infusion in control and TgSR+/− mice. Mice were intravenously infused with DiI-acetylated human LDL and sacrificed after 10 minutes. Liver pieces were embedded in O.C.T., 3 to 5 μM slices prepared and viewed by fluorescent microscopy using a rhodamine filter set. Top panel shows a control mouse hepatic section demonstrating nonparenchymal cell to DiI uptake evidenced by fluorescence being confined to elongated cells surrounding sinusoids. The bottom panel shows a section from a TgSR+/− mouse. In addition to DiI uptake by nonparenchymal cells, extensive dye uptake occurred in polyhedral-shaped parenchymal cells (donut-shaped cells) as evidenced by the perinuclear staining. Sinusoidal cells, arrows; parenchymal cell nucleus, N.

FIG. 5

Clearance of ¹²⁵I-acetylated-hLDL in control and TgSR+/− mice. Five TgSR+/− (●) and five control (&Ovalhollow;) mice were tail vein injected with ¹²⁵I-ac-hLDL. Blood samples were collected periodically up to 8 minutes to determine plasma radioactivity clearance. To control for nonscavenger receptor mediated ¹²⁵I-ac-hLDL clearance, three TgSR+/− (▪) and three control [symbol] mice were coinjected with 0.1 mL of ¹²⁵I-ac-hLDL preparation plus 0.1 mL Fucoidan. Radioactivity data are expressed as percent of the first 20-second time point. Each data point represents data averaged from five (¹²⁵I-ac-hLDL alone) or three mice (¹²⁵I-ac-hLDL plus Fucoidan).

FIG. 6

Lipoprotein cholesterol analysis of plasma from nontransgenic control (FVB×C57BL/6J), TgSR+/−, and TgSR+/+ mice maintained on chow or fed the HFHC diet for up to 3 weeks. High performance gel-filtration chromatographic lipoprotein profile analysis of 10 μL plasma from these mice was determined weekly as described in Example 11. A blood sample from TgSR+/+ Mouse 242 on chow was not obtained, and control Mouse 148 died from anesthesia overdose at 2 weeks. Although not shown on the figure the peak height (Y-axis)=OD 490, and is the same scale for each of the 12 groups shown.

FIG. 7

Plasma cholesterol in apo B containing lipoproteins (Top Panel), HDL (Middle Panel), and the apo B containing lipoprotein to HDL cholesterol ratio (lower panel) in nontransgenic control (●, n=4 or 5), TgSR+/− (▴, n=5), and TgSR+/+ (▪, n=3 or 4) mice maintained on chow or fed the HFHC diet for up to 3 weeks. Total plasma cholesterol (Table I) and lipoprotein profiles from data shown in FIG. 6 were used for the determinations. Data points represent the mean±SEM.

FIG. 8

Hepatic lipid analysis in TgSR+/− mice. In the top panel, typical livers from control and TgSR+/− mice maintained on chow diets showed no evidence of the fatty accumulation. Livers from control mice fed the HFHC diet were always white indicative of a fatty liver, while livers from TGSR+/− mice fed the HFHC diet appeared only slightly discolored. The lower panel shows hepatic lipid analysis from the four control and five TgSR+/− mice after 3 weeks on the high-fat, high-cholesterol diet (i.e., from animals studied in Table I, FIG. 6 and 7). Data represent the mean± SEM. Significance difference in mean was determined by a Student's t-test for unpaired data.

FIG. 9

Total fecal bile acids were determined weekly in five control [symbol] and five TgSR+/− (▪) mice fed chow, and then the HFHC diet for 3 weeks as described in Example 13. Data represent the mean±SEM.

FIG. 10

Two control and two heterozygous SR transgenic mice were fed the high-fat, high-cholesterol diet for 3 weeks. Hepatic total RNA (10 μg/lane) were run on duplicate gels, blotted and probed for mouse 7α-hydroxylase or mouse actin as described in Example 3. The 7α-hydroxylase to actin ratio was elevated 2-fold in the SR transgenic mice. Data represent the average of 2 mice per group.

### DETAILED DESCRIPTION OF THE INVENTION

The term “transient expression” means the expression of a transfected gene that is temporary, usually lasting only a few days to a few weeks.

The term “stable expression” means the expression of a transfected gene where the expression is sustained.

The term “mammal” includes humans.

The term “liver specific promoter” means a promoter constructed of either homologous or heterologous promoter elements either naturally occurring or artificially, including synthetically created.

The term “partial minigene” means a minigene lacking one or more elements outside the coding sequence such as, for example, a promoter, a 5′ untranslated region, a 3′ untranslated region, a polyadenylation signal, and the like.

In order to directly determine whether or not hepatic SRs have a protective anti-atherosclerotic role, transgenic mice overexpressing hepatic bovine SR Type I were created in the genetic background of the FVB mouse crossed to the atherosclerosis susceptible C57BL/J6 mouse. Both heterozygous (TgSR+/−) and homozygous (TgSR+/+) mice were created. Uptake of modified lipoproteins was greatly enhanced in the liver of these animals. Furthermore, when fed cholesterol-enriched diets, these mice present with marked reductions in apo B-containing lipoproteins and hepatic cholesteryl esters, and increased hepatic 7α-hydroxylase mRNA levels and total fecal bile acids. These data directly demonstrate a potential in vivo anti-atherosclerotic role of hepatic scavenger receptors.

Creation of SR Transgenic Mice

To create mice with hepatic expression of the bovine SR Type I, a SR minigene containing the mouse transferrin promotor was constructed (FIG. 1). Based on the work of Kozak, et al. (Kozak M., supra., 1986; Kozak M., supra., 1989), 5 bp of the untranslated region of the bSR cDNA sequence (Idzerda R. L., Behringer R. R., Theisen M., Huggenvik J. I., McKnight G. S., Brinster R. L., supra., 1989) was incorporated into the construct since inclusion of this element should faciltate correct initiation and highly efficient translation of the SR. In our experiments this concept was not examined rigorously in that we did not construct nor test a minigene lacking these 5 bp. The SR minigene was injected into hybrid fertilized eggs obtained from a C57BL/6J female crossed to a FVB male. PCR and Southern blotting indicated three potential transgenic mice were created (data not shown). These mice were breed to C57BL/6J×FVB; offspring from these crosses indicated that out of the three potential founders, two were chimerics and one had transgene integration into the germline. Southern blot results suggested approximately 30 copies of the transgene were present per cell. In some studies, TgSR+/− were crossed to generate homozygous mice (TgSR+/+).

Expression of the Bovine SR in Transgenic Mice

Tissue-specific expression of bovine SR mRNA was examined by RT-PCR and Northern blot analysis of total RNA isolated from tissue of TgSR+/− and nontransgenic controls (C57BL/6J×FVB). By RT-PCR a 1 kb cDNA fragment was amplified in a TgSR+/− but not in a control mice (FIG. 2), demonstrating the presence of bovine SR mRNA in the TgSR+/− mouse liver. Bovine SR mRNA was predominantly expressed in liver with a much smaller amount found in kidney. A minute amount of bovine SR expression was also observed in brain (FIG. 2). We estimate, hepatic mRNA levels of the bovine SR to be approximately 20- to 30-fold higher than the endogenous mouse SR (data not shown).

Detergent solubilized nonreduced liver membrane preparations from the TgSR+/− mice revealed the presence of monomeric plus possibly monomeric precursors (up to ˜80 kDA), dimeric (˜160 kDa), and trimeric (˜240 kDa) forms of the bovine SR by Western blotting (FIG. 3).

Hepatic Parenchymal and Nonparenchymal Expression of the Bovine SR

Histological examination of liver sections following intravenous infusion of fluorescent DiI-acetylated human LDL in control and TgSR+/− mice indicates the presence of the fluorescence probe in both nonparenchymal Kupffer and sinusoidal cells (FIG. 4). However, unlike nontransgenic mouse, TgSR+/− mouse liver parenchymal cells were fluorescent suggesting these cells expressed the transgene (FIG. 4).

Fractional Catabolism of ¹²⁵I-Acetylated LDL in SR Transgenic Mice

The fractional catabolism of ¹²⁵I-ac-hLDL was determined in five TgSR+/− and five nontransgenic littermates. Mice were tail vein injected with the probe and 10 μL sinus orbital bleeds were periodically taken up to 8 minutes. The t½ for ¹²⁵I-ac-hLDL clearance in the TgSR+/− was 2.5 times faster (75 seconds) than in control mice (186 seconds) (FIG. 5). In three TgSR+/− and three nontransgenic littermates simultaneously injected with both Fucoidan and ¹²⁵I-ac-hLDL, the SR mediated clearance of the probe was blocked (FIG. 5).

Plasma Lipids and Lipoprotein Profiles

Weekly plasma triglycerides and total cholesterol from control, TgSR+/−, and TgSR+/+ mice initially on a chow diet then maintained on a HFHC diets for 3 weeks are shown in Table I. In all mice and under all dietary conditions plasma triglycerides were similar (Table I) on chow, basal cholesterol levels were similar in control and transgenic mice. When fed the high-fat, high-cholesterol diet, total plasma cholesterol rose in all mice. However, at Week 3, total plasma cholesterol in the TgSR+/− and TgSR+/+ mice increased to only 59% and 83%, respectively, of that observed in the control mice. High performance gel-filtration chromatographic lipoprotein profile analysis of plasma from these mice (FIG. 6) was utilized to determine the distribution of cholesterol between lipoproteins (FIG. 7). On the chow diet lipoprotein cholesterol profiles were similar in control and SR transgenic mice; HDL carried the majority of cholesterol under these conditions (FIGS. 6 and 7). When fed the HFHC diet, cholesterol predominantly rose in apo B containing lipoproteins relative to HDL in control mice (FIGS. 6 and 7). In both TgSR+/− and TgSR+/+ mice, apo B containing lipoproteins, rose to only half the amount observed in the control mice (FIGS. 6 and 7). In the TgSR+/− mice, HDL rose more rapidly than the controls, however, after 3 weeks on the HFHC diet HDL cholesterol levels converged (FIG. 7). In contrast, in the TgSR+/+ mice fed the HFHC diet, HDL cholesterol continued to rise and the level was significantly greater than control mice levels at 3 weeks (FIGS. 6 and 7). These marked differences in lipoprotein profiles can be appreciated as the ratio of apo B-containing lipoprotein cholesterol to that of HDL cholesterol (FIG. 7). Thus, in the TgSR+/− and TgSR+/+ mice this ratio rose 2.8-fold with the HFHC diet, while this ratio rose to 6.6-fold in the control mice.

TABLE I

| | | | | | | Cholesterol | |
|---|---|---|---|---|---|---|---|
| | | | Cholesterol (mg/dL) | | | Ratio | Triglyceride |
| Diet^{a,b,c} | Genotype | N | Total | VLDL + IDL + LDL | HDL | VLDL + IDL + LDL/HDL | (mg/dL) |
| Chow Diet | Control | 5 | 60 ± 7 | 15 ± 3 | 45 ± 4 | 0.326 ± 0.031 | 49 ± 3 |
| | TgSR+/− | 5 | 59 ± 6 | 14 ± 2 | 45 ± 5 | 0.318 ± 0.021 | 51 ± 10 |
| | TgSR+/+ | 3 | 78 ± 7 | 21 ± 2 | 56 ± 5 | 0.383 ± 0.019 | 56 ± 4 |
| Chow Diet Differences^{d} | | | | | | | |
| Control vs TgSR+/− | | | NS | NS | NS | NS | NS |
| Control vs TgSR+/+ | | | NS | NS | NS | NS | NS |
| TgSR+/− vs TgSR+/+ | | | NS | NS | NS | NS | NS |
| 1 Week High-Fat, | Control | 5 | 203 ± 37 | 132 ± 28 | 71 ± 10 | 1.800 ± 0.204 | 52 ± 6 |
| High-Cholesterol | TgSR+/− | 5 | 174 ± 17 | 70 ± 10 | 105 ± 8 | 0.656 ± 0.041 | 60 ± 7 |
| Diet | TgSR+/+ | 4 | 180 ± 15 | 77 ± 13 | 104 ± 7 | 0.752 ± 0.133 | 55 ± 6 |
| 1 Week High-Fat, High- | | | | | | | |
| Cholesterol Diet Differences | | | | | | | |
| Control vs TgSR+/− | | | NS | 0.0045 | 0.0084 | <0.0001 | NS |
| Control vs TgSR+/+ | | | NS | 0.0164 | 0.0155 | <0.0001 | NS |
| TgSR+/− vs TgSR+/+ | | | NS | NS | NS | NS | NS |
| 2 Weeks High-Fat, | Control | 5 | 242 ± 26 | 155 ± 19 | 88 ± 8 | 1.768 ± 0.136 | 39 ± 3 |
| High-Cholesterol Diet | TgSR+/− | 5 | 190 ± 15 | 85 ± 8 | 105 ± 10 | 0.828 ± 0.098 | 53 ± 19 |
| | TgSR+/+ | 4 | 256 ± 21 | 99 ± 8 | 157 ± 16 | 0.650 ± 0.055 | 52 ± 2 |
| 2 Weeks High-Fat, High- | | | | | | | |
| Cholesterol Diet Differences | | | | | | | |
| Control vs TgSR+/− | | | NS | 0.0018 | NS | <0.0001 | NS |
| Control vs TgSR+/+ | | | NS | 0.0165 | <0.0001 | <0.0001 | NS |
| TgSR+/− vs TgSR+/+ | | | 0.0319 | NS | 0.0003 | NS | NS |
| 3 Weeks High-Fat, | Control | 4 | 323 ± 35 | 226 ± 29 | 96 ± 7 | 2.340 ± 0.204 | 38 ± 2 |
| High-Cholesterol Diet | TgSR+/− | 5 | 192 ± 17 | 93 ± 14 | 100 ± 11 | 1.004 ± 0.240 | 27 ± 1 |
| | TgSR+/+ | 4 | 270 ± 13 | 132 ± 15 | 138 ± 11 | 0.998 ± 0.181 | 86 ± 27 |

| | | | | | |
|---|---|---|---|---|---|
| 3 Weeks High-Fat, High-Cholesterol | | | | | |
| Diet Differences | | | | | |
| Control vs TgSR+/− | <0.0001 | <0.0001 | NS | <0.0001 | NS |
| Control vs TgSR+/+ | NS | 0.0002 | 0.0041 | <0.0001 | 0.0047 |
| TgSR+/− vs TgSR+/+ | 0.013 | 0.0842 | 0.0057 | NS | 0.0003 |

^{a}Plasma lipids and lipoprotein levels in control, heterozygous transgenic bovine SR (TgSR+/−), and homozygous transgenic bovine SR (TgSR+/+) mice.
^{b}Blood samples were obtained following an 8-hour fast from mice on the chow diet; and after 1, 2, and 3 weeks on the high-fat, high-cholesterol diet.
^{c}Data represent mean ± SEM.
^{d}ANOVA, Fisher's PSLD posthoc analysis, NS = not significant (significance level = 5%), all other as p-values.

Cholesterol Absorption and Food Intact Studies

Diminished total plasma cholesterol in the transgenic mice could possibly reflect a reduced food intake or an impeded cholesterol absorption. Food intake was, therefore, recorded over a 3-week period for five control and five TgSR+/− on the HFHC diet. Average body weight for each group was 22 g. Weekly food intake was virtually identical between groups; control mice consumed 23.1, 22.9, and 24.3 g/week, while the TgSR+/− mice consumed 21.9, 27.5, and 24.7 g/week, for the first, second, and third week, respectively.

Next, cholesterol absorption was determined in three control and five TgSR+/− mice. Animals were oral gavaged with a ³H-cholesterol/¹⁴C-β-sitosterol in sunflower oil, placed on the HFHC diet and feces were collected for 4 days. The ³H/¹⁴C ratio in the oral dose and in the neutral lipid fraction extracted from the feces was utilized to estimate the amount of cholesterol absorbed. The percent cholesterol absorption was similar in control (56.8±3.4) and TgSR+/− (56.6±4.4) mice.

Overall these studies suggest the diminished levels of plasma cholesterol observed in the SR transgenic mice is not the result of reduced food intact or cholesterol absorption.

Hepatic Lipids

Gross visual examination of control and TgSR+/− livers from mice maintained on chow diets showed no evidence of fatty accumulations. Livers of control mice fed the HFHC diet showed considerable fat accumulation. In contrast, livers from TgSR+/− mice fed the HFHC diet appeared either normal (not shown) or only slightly discolored (shown) (FIG. 8). To determine whether hepatic lipids would accumulate in the SR transgenic mice, lipid analysis was performed on the four control and five TgSR+/− mice after 3 weeks on the high-fat, high-cholesterol diet (i.e., from animals studied in Table I, FIGS. 6 and 7). In the TgSR+/− mice, hepatic cholesteryl esters, triglycerides, and nonesterified cholesterol did not accumulate, but were instead significantly reduced by 59%, 61%, and 36%, respectively (FIG. 8). Hepatic phosphatidylethanolamine and phosphatidylcholine levels were similar (FIG. 8).

Fecal Bile Acids

To determine whether there would be an increase flux of bile acids in the SR transgenic mice, total fecal bile acids were determined weekly in five control and five TgSR+/− mice fed chow 1 week, and the HFHC diet for 3 weeks. On chow, fecal bile acids were similar in control (1.51±0.20 mg/week) and TgSR+/− (1.37±0.04 mg/week) mice. On the HFHC diet, fecal bile acids markedly increased 5.4-fold by 1 week (8.19±0.95 mg/week) and remained constant throughout the study (Week 2, 8.06±1.65 mg/week; Week 3, 8.49±0.36 mg/week). Similarly, the fecal bile acids TgSR+/− mice fed the HFHC diet increased 5.8-fold in the first week (7.91±1.54). In contrast, however, fecal bile acids in the subsequent 2 weeks progressively increased (Week 2, 9.23±1.17 mg/week; Week 3, 10.83±0.33 mg/week) (FIG. 9).

Hepatic 7α-Hydroxylase mRNA Levels

To determine if messenger RNA levels for 7α-hydroxylase (the rate-limiting enzyme for hepatic bile acid synthesis) were elevated to a greater extent in the TgSR+/− mice, total hepatic RNA was extracted from two control and two TgSR+/− mice maintained on the HFHC diet for 3 weeks. Northern blot analysis demonstrates 7α-hydroxylase mRNA levels relative to mouse actin mRNA were elevated 2-fold in the TgSR+/− compared to control mice (FIG. 10).

Thus, when the TgSR mice were fed an atherogenic diet, we observed neither a difference in food intake nor in absorption of cholesterol. However, their plasma lipoprotein profiles showed reduced accumulation of apo B containing lipoprotein cholesterol. This effect was quite dramatic. TgSR+/− mice showed almost a 2-fold reduction in the rise of apo B containing lipoproteins after a week on the HFHC diet as compared to the nontransgenic mice. This differential response was consistent throughout the 3-week feeding period. This was in sharp contrast to the normal chow feeding period, in which the nontransgenic and transgenic mice maintained virtually equivalent lipoprotein profiles. Furthermore, when the TgSR+/− mice were on the HFHC diet, a compensatory rise in hepatic cholesterol was not observed; in fact, both hepatic cholesterol and cholesteryl esters were reduced in the transgenic mice. These data suggested an enhanced secretion of biliary cholesterol as bile acids. Indeed, both hepatic 7α-hydroxylase mRNA levels and total fecal bile acids were elevated in the transgenic mice. Overall, these studies suggest that the overexpressing of the hepatic SR enhanced the flux of cholesterol secretion.

Based on our Northern blotting experiments in tissues from TgSR mice, SR mRNA expression was indeed confined predominantly to the liver. Hepatic fluorescent microscopy of the TgSR+/− mice injected with DiI-acetylated-LDL demonstrated SR activity in the sinusoidal endothelial cells, which normally express SR (Dresel H. A., Friedrich E., Via D. P., Sinn H., Ziegler R., Schettler G., supra., 1987; van Berkel T. J. C., Nagelkerke J. F., Kruijt J. K., supra., 1981; Dresel H. A., Friedrich E., Via D. P., Schettler G., Sinn H., supra., 1985; de Rijke Y. B., van Berkel T. J. C., supra., 1994; Nagelkerke J. F., Barto K. P., van Berkel T. J. C., supra., 1983; Pitas R. E., Boyles J., Mahley R. W., Montgomery B. D., supra., 1985; Horiuchi S., Takata K., Maeda H., Morino Y., supra., 1985; van Berkel T. J. C., de Rijke Y. B., Kruijt J. K., supra., 1991; Esbach S., Pieters M. N., Van der Boom J., et al., supra., 1993) and also in hepatocytes in which SR are normally almost undetectable (Nagelkerke J. F., Barto K. P., van Berkel T. J. C., supra., 1983). Furthermore, our observation of a 2.5-fold enhanced clearance rate for ac-hLDL in the transgenic mice suggests a hepatic-directed clearance which affords a protective effect for atherosclerosis.

As has been suggested by the early studies from the laboratory of Brown and Goldstein (Brown M. S., Goldstein J. L., supra., 1990), SR have been hypothesized to play a protective role in atherogenesis by removing modified lipoproteins. Indeed, apo B containing lipoproteins from rabbits fed high-cholesterol diets are more susceptible to Cu⁺²-induced modification than LDL isolated from control rabbits in vitro (Nenseter M. S., Gudmundsen O., Malterud K. E., Berg T., Drevon C., Biochim. Biophys. Acta., 1213:207-214 (1994)). Furthermore, studies of Palinski, et al. (Palinski W., Rosenfeld M. E., Ylä-Herttuala S., et al., Proc. Natl. Acad. Sci. USA, 86:1372-1376 (1989)), have provided evidence for the in vivo oxidative modification of LDL in LDL-receptor-deficient rabbits. Furthermore, studies of Palinski, et al. (Palinski W., Rosenfeld M. E., Ylä-Herttuala S., et al., supra., 1989; Palinski W., Ord V. A., Plump A. S., Breslow J. L., Steinberg D., Witztum J. L., Arterioscler. Thromb., 14:605-616 (1994)), utilizing LDL-receptor deficient rabbits (Palinski W., Rosenfeld M. E., Ylä-Herttuala S., et al., supra., 1989) or apo E deficient-mice (Palinski W., Ord V. A., Plump A. S., Breslow J. L., Steinberg D., Witztum J. L., supra., 1994; Plump A. S., Smith J. D., Hayek T., Cell, 71:343-353 (1992)), have provided in vivo evidence for the oxidative modification of apo B containing lipoproteins by demonstrating the presence of high titers of autoantibodies to malondialdehyde-lysine, an epitope that presents on “modified” lipoproteins. Since significant quantities of “modified” apo B containing lipoproteins may also be formed in mice fed the HFHC diet, overexpression of the SR is likely responsible for their reduction, characterized by reduced amounts of apo B containing lipoprotein cholesterol. This hypothesized premise suggests that the SR expressed in vivo are exquisitely sensitive to slight modifications of lipoproteins, since these “modified” lipoproteins cannot be shown to accumulate in hypercholestolemic plasmas. Furthermore, since “modified” lipoproteins are not observed in plasma it is also likely the capacity of SR is not exceeded. However, a competition between arterial subendothelial SR with those of liver likely exists. Thus, under conditions where hepatic SR expression is high, “modified” lipoproteins would be less likely to bind SR present in the aortic subendothelium. However, in certain pathophysiological or procedural-induced conditions (e.g., atherectomy, angioplasty), the arterial endothelium becomes compromised and the relative number and assess to subendothelial SR increases. If such a situation occurs in mammals, including humans, the modified lipoproteins would kinetically favor binding to the SRs expressed by cells in the subendothelium and could lead to enhanced arterial lipid deposition.

With respect to reduction of apo B containing lipoproteins we did not observe a gene dosage effect between the TgSR+/− and the TgSR+/+ mice. Possibly, sufficient SRs are produced in the heterozygous animals to efficiently remove all modified lipoproteins that form in these mice. The elevated rise in HDL was unexpected. The observation that HDL rose to a greater extent in the TgSR+/+ mice, or earlier in the TgSR+/− and TgSR+/+ mice suggested alterations in HDL metabolism. The explanation for this finding is not entirely clear and is cause for some speculation. Possibly, the catabolism of HDL is diminished in these mice. This may occur due to an increase removal of apo E with apo B containing particles, possibly reducing the apo E pool necessary for whole HDL particle clearance (Bisgaier C. L., Siebenkas M. V., Williams K. J., J. Biol. Chem., 264:862-866 (1989)). Alternatively, HDL production may be enhanced in these mice. This may occur due to increased expression of the SR. Possibly, elevated amounts of “modified” VLDL remnants are marginated within the liver due to the increased amounts of SR. The triglyceride and phospholipid of the trapped remnants, as well as circulating VLDL remnants and HDL, are hepatic lipase substrates (Jackson R. L., B. P. New York, 141-181 (1983)). Unlike other species liver-derived hepatic lipase in mice is not anchored to liver membrane glycosaminoglycan but freely circulates (Peterson J., Bengtsson-Olivercrona G., Olivecrona T., Biochim. Biophys. Acta., 878:65-70 (1986)). Therefore, increased levels of this enzyme may be sequestered near its site of synthesis due to the increased presence of bound “modified” VLDL remnant substrate to the SR receptors. Enhanced lipolysis of these modified “remnants” by hepatic lipase would lead to generation of redundant surface phospholipid that could potentially elevate production of the HDL pool (Tall A. R., Small D. M., N. Engl. J. Med., 299:1232-1236 (1978); Eisenberg S., Patsch J. R., Sparrow J. T., Gotto A. M. Jr., Olivecrona T., J. Biol. Chem., 254:12603-12608 (1979); Schaefer E. J., Wetzel M. G., Bengtsson G., Scow R. O., Brewer H. B. Jr., Olivecrona T., J. Lipid Res., 23:1259-1273 (1982); Tam S. P., Breckenridge W. C., J. Lipid Res., 24:1343-1357 (1983)). Since mice lack cholesteryl ester transfer protein (Agellon L. B., Walsh A., Hayek T., et al., J. Biol. Chem., 260:10796-10801 (1990)), HDL triglyceride cannot be efficiently derived from VLDL and VLDL remants by exchange with HDL cholesteryl esters (Tall A. R., J. Lipid Res., 34:1255-1274 (1993)), therefore, expansion of the particles' nonpolar core will be largely due to cholesteryl ester accumulation. Since HDL phospholipid surface are also substrate for hepatic lipase, and if this enzyme is largely sequestered in liver due to the increase presence of bound “modified” particles, a secondary effect would be reduced levels of circulating hepatic lipase. Therefore, an altered HDL catabolism might develop. Possibly, the phospholipid surface of these particles might not be subject to extensive lipolysis, which could allow these HDL to be better substrates for lecithin:cholesterol acyl transferase resulting in accumulation of core cholesteryl esters.

The SR gene can be introduced into cells by any of the many methods known for introducing DNA into cells, either transiently or stably (“Gene Therapeutics” Methods and Applications of Direct Gene Transfer, Wolff, J. A., ed., Birkhäuser, Boston, 1994; Kozarsky, K. F., McKinley, D. R., Austin, L. L., Raper, S. E., Stratford-Perricaudet, L. D., Wilson, J. M., J. Biol. Chem 269:13695-13702 (1994); Henry, J. and Gerard, R. D., Proc. Natl. Acad. Sci. USA 90:2812-2816 (1993); Archer, J. S., Hennan, W. S., Gould, M. N., Bremel, R. D., Proc. Natl. Acad. Sci. USA 91:6840-6844 (1994); Wolff, J. A., Malone, R. W., Williams, P., Chang, W., Acsadi G., Jani, A., Felgner, P. L., Science 247:1465-1468 (1990); Wolff, J. A., Williams, P., Ascadi, G., Jiao, S., Chong, W., Biotechniques 11:474-485 (1991); Barr, E. and Leiden, J. M., TCM 4:57-62 (1994); Kozarsky, K., Grossman, M., Wilson, J. M., Somatic Cell and Molecular Genetics 19:449-458 (1993); Wu, C. H., Wilson, J. M., Wu, G. Y., J. Biol. Chem. 264:16985-16987 (1989); Ishibashi, S., Brown, M. S., Goldstein, J. L., Gerard, R. D., Hammer, R. E., Herz, J., J. Clin. Invest. 92:883-893 (1993); Liu, T. J., Kay, M. A., Darlington, G. J., Woo, S. L., Somatic Cell and Molecular Genetics 18:89-96 (1992); Kay, M. A., Li, Q., Liu, T. J., Leland, F., Toman, C., Finegold, M., Woo, S. L., Hum. Gene Ther. 3:641-647 (1992); Kay, M. A., Ponder, K. P., Woo, S. L., Breast Cancer Res. Treat, 21:83-93 (1992); Chen, S. H., Shine, H. D., Goodman, J. C., Grossman, R. G., Woo, S. L., Proc. Natl. Acad. Sci USA 91:3054-3057 (1994); Kolodka, T. M., Finegold, M., Woo, S. L., Somatic Cell and Molecular Genetics 19:491-497 (1993)). The methods for introducing DNA into cells include calcium phosphate coprecipitation, cationic liposomes, electroporation, receptor mediated endocytosis, particle-mediated gene transfer, attachment to synthetic peptides, or for some cell types, naked DNA can be used. The SR genes can also be introduced by any of the well-known viral vectors, including retroviruses, adenovirus, adeno-associated virus, and herpes viruses. Thus, the SR gene of the present invention can be introduced into cells by conventional gene transfer technology known to those skilled in the art.

The use of the SR to attenuate hypercholesterolemia and its pathological sequelae in the form of gene therapy proceeds as follows. The SR minigene construct is prepared using either a viral or nonviral method of delivery. The formulation could be, for example, using cationic liposomes (Philip B., et al., J. Biol. Chem., 268:16087-16090 (1993)) where 10 μg to 10 mg of a vector expressing the scavenger receptor is delivered. For in vivo administration, it will usually be preferred to use a vector that will direct tissue-specific gene expression to the liver. The resulting preparation is infused intravenously into candidate patients, and the efficacy of treatment is monitored by measuring the patient's plasma cholesterol and its distribution among lipoproteins. Alternatively, the treatment is carried out ex vivo. A portion of the patient's liver is surgically removed. Liver parenchymal cells are isolated by standard techniques and placed in tissue culture. The liver cells are then transfected with the SR gene by standard techniques, placed in culture for several days, and tested for the cell surface expression of the SR. The resulting cell preparation is then reinfused into the patient wherein the liver cells take up residence in the liver and express the SR. Efficacy of treatment is monitored by measuring plasma total cholesterol and its distribution among lipoproteins. Optimal treatment of a patient receiving SR gene therapy will often involve coadministration with an ACAT inhibitor; a HMG-CoA reductase inhibitor, a bile acid sequestrant, or a lipid regulator.

Examples of ACAT inhibitors include DL-melinamide disclosed in British Patent 1,123,004 and Japan. J. Pharmacol, 42:517-523 (1986); 2,2-dimethyl-N-(2,4,6-trimethoxyphenyl)dodecanamide disclosed in U.S. Pat. No. 4,716,175; N-[2,6-bis(1-methylethyl)phenyl]-N′-[[1-(4-dimethylaminophenyl)cyclopentyl]methyl]urea disclosed in U.S. Pat. No. 5,015,644; 2,6-bis(1-methylethyl)phenyl[[2,4,6-tris(1-methylethyl)phenyl]acetyl]sulfamate disclosed in copending U.S. patent application Ser. No. 08/233,932 filed Apr. 13, 1994; and the like. U.S. Pat. Nos. 4,716,175 and 5,015,644 and U.S. patent application Ser. No. 08/233,932 and British Patent 1,123,004 and Japan. J. Pharmacol, 42:517-523 (1986) are hereby incorporated by reference.

Examples of HMG-CoA reductase inhibitors include lovastatin disclosed in U.S. Pat. No. 4,231,938; pravastatin disclosed in U.S. Pat. No. 4,346,227; simvastatin disclosed in U.S. Pat. No. 4,444,784; fluvastatin disclosed in U.S. Pat. No. 4,739,073; atorvastatin disclosed in U.S. Pat. Nos. 4,681,893 and 5,273,995; and the like. U.S. Pat. Nos. 4,231,938, 4,346,227, 4,444,784, 4,681,893, 5,273,995, and 4,739,073 are hereby incorporated by reference.

Examples of bile acid sequestrants include colestipol disclosed in U.S. Pat. Nos. 3,692,895 and 3,803,237; cholestyramine disclosed in U.S. Pat. No. 3,383,281 and R. Casdorph in Lipid Pharmacology 2:222-256, Paoletti C., Glueck J., eds. Academic Press, NY 1976; and the like. U.S. Pat. Nos. 3,692,895, 3,803,237, and 3,383,281 and R. Casdorph, supra, are hereby incorporated by reference.

Examples of lipid regulators include gemfibrozil described in U.S. Pat. No. 3,674,836; bezafibrate disclosed in U.S. Pat. No. 3,781,328; clofibrate disclosed in U.S. Pat. No. 3,262,850; fenofibrate disclosed in U.S. Pat. No. 4,058,552; niacin disclosed in McElvain, et al., Org. Syn., 4:49 (1925); and the like. U.S. Pat. Nos. 3,674,836, 3,781,328, 3,262,850, and 4,058,552 and McElvain, et al., Org. Syn., 4:49 (1925) are hereby incorporated by reference.

The following nonlimiting examples illustrate the inventor's preferred methods for preparing a SR gene of the present invention.

### EXAMPLE 1

Bovine SR Minigene Preparation

A partial SR Type I cDNA clone was isolated from a bovine lung λgt10 cDNA library (Clontech Laboratories, Inc., Palo Alto, Calif.) using three oligonucleotides that were selected based on the published sequence (Kodama T., Freeman M., Rohrer L., Zabrecky J., Matsudaira P., Kreiger M., Nature, 343:531-535 (1990)). This cDNA fragment, 1.8 kb in length, was subcloned into pGEM 3Zf (−) (Promega Corp, Madison, Wis.). The missing 0.3 kb of the 5′ end of the partial cDNA clone was synthesized by coupled reverse transcriptase and polymerase chain reaction (PCR) (Mullis K. B., Faloona F. A., Methods in Enzymology, 155:335-350 (1987); Saiki R. K., Gelfand D. H., Stoffel S., Science, 239:487-491 (1988)) using bovine lung mRNA (Clontech Laboratories, Inc.) as a template and the specific 5′ (5′-GGGCGTCCGGATTTGGAGATATATCTGCA-3′) and 3′ (5′-GCGGATCCGAAGTATGGC-ACGTGGGATGACTTTCC-3′) primers. This cDNA generated fragment was then ligated into the pGEM 3Zf (−) clone (Promega Corp, Madison, Wis.) that contained 1.8 kb of bovine SR between BamHI in the plasmid polylinker site and the SR sequence internal AccIII restriction site. The full length bovine SR cDNA was verified (Kodama T., Freeman M., Rohrer L., Zabrecky J., Matsudaira P., Kreiger M., supra., 1990) by nucleotide sequencing using the dideoxy-chain termination method (Sanger F., Nicklen S., Coulson A. R., Proc. Natl. Acad. Aci. USA, 74:5463-5467 (1977)). To construct the bovine SR minigene approximately 3 kb of the mouse transferrin promoter (Idzerda R. L., Behringer R. R., Theisen M., Huggenvik J. I., McKnight G. S., Brinster R. L., Mol. Cell. Biol., 9:5154-5162 (1989)) was ligated to the 5′ end of the bovine SR cDNA. The mouse transferrin promoter contained an artificially introduced BamHI restriction site (Idzerda R. L., Behringer R. R., Theisen M., Huggenvik J. I., McKnight G. S., Brinster R. L., supra., 1989) at the 3′ end which was convenient for ligation to the bovine SR clone. The resulting construct contained 5 bp of the 5′ untranslated region of the bovine SR upstream of the ATG start site. Inclusion of this short 5 bp untranslated region in the construct appears to be necessary for efficient translation (i.e., “first AUG rule”) (Kozak M., supra., 1986; Kozak M., supra., 1989). At the 3′ end of the promoter-bovine SR construct, 0.65 kb of the human growth hormone gene sequence containing the stop signal was ligated at a Asp700/SmaI fusion site (FIG. 1). The total size of the minigene construct was 5.2 kb and was isolated by cutting with EcoRI (5′ end) and NotI (3′ end), purified with Qiaex (Qiagen Inc., Chatsworth, Calif.) and utilized for production of transgenic mice.

### EXAMPLE 2

Production of Bovine SR Transgenic Mice

Fertilized one-cell embryos were isolated from superovulated C57BL/6J×FVB mice (Jackson Laboratories). To create transgenic mice, approximately 1000 male pronuclei of the fertilized embryos were microinjected with the purified 5.2 kb minigene construct described above at a DNA concentration of 3 ng/μL (Brinster R. L., Palmiter R. D., The Harvey Lectures, Series 80:1-38 (1980); Hogan B., Costantini F., Lacy E., Cold Spring Harbor Laboratory. New York, 1986) and reimplanted into ICR pseudo-pregnant mice. Forty-five potential founders were screened by Southern blotting and PCR (see below). Of these, three mice were positive and, therefore, breed to C57BL/6J mates. Of the three founders, only one female mouse (Mouse 1876) incorporated the transgene in the germline and passed it on to offspring; the other two potential founders were chimerics. A heterozygous line (TgSR+/−) was established by breeding Mouse 1876 to nontransgenic C57BL/6J mice. Homozygous mice (TgSR+/+) were obtained by crossing TgSR+/−. Both TgSR+/− appeared healthy and thrive for at least 2 years and the TgSR+/+ have been healthy since their creation (approximately 0.5 years).

Bovine SR minigene transmission in founder and offspring generations was confirmed by both Southern blot analysis (Southern E. M., J. Mol. Biol., 98:503-517 (1975)) and PCR. For Southern blot analysis, genomic DNA (10 μg) was digested with restriction enzymes EcoRI and BamHI or BamHI alone. The samples were electrophoresed in a 1% agarose gel and blotted onto Zetaprobe membranes (Bio-Rad, Laboratories, Hercules, Calif.). Blots were prehybridized for 5 to 6 hours at 42° C., and then hybridized overnight to a 0.7 kb fragment (see FIG. 1) that was random primed (Boeringer Mannheim, Indianapolis, Ind.) using ³²P-dCTP (Amersham Corp, Arlington Heights, Ill.). By PCR analysis, using the bovine specific primers 5′-CCTCCATCCAGGAACATGAG-3′ and 5′-CCTTTTCTGTGGATAAAATTC-3′, a 1 kb cDNA fragment could be amplified from transgenic mice but not from nontransgenic littermates.

Southern blot analysis was used to estimate transgene copy number. TgSR+/− genomic DNA hybridization intensities were compared to standards comprised of control mouse genomic DNA containing variable amounts of the bovine SR minigene DNA.

### EXAMPLE 3

RNA Analysis

Tissue specific expression of bovine SR, mouse 7α-hydroxylase and mouse actin (Ambion, Inc., Austin, Tex.) mRNA were determined by Northern blot analysis. Total RNA was isolated from liver, spleen, lung, brain, heart, kidney, small intestine, large intestine, ovary, adipose, and muscle from control and transgenic mice with RNAzol (Biotecx Laboratories, Inc., Houston, Tex.) according to instructions supplied with reagent. Quantitative and qualitative assessment of total RNA were determined spectrophotometrically and on 1% analytical agarose gels, respectively.

Before performing Northern blot analysis total liver RNA (5 μg) from TgSR+/− and nontransgenic littermates were used for reverse transcriptase reactions utilizing the upstream specific bovine SR primer (5′-CCTTTTCTGTGGATAAAATTC-3′) and a first strand cDNA synthesis kit (Superscript, BRL). A control reaction without the reverse transcriptase enzyme (Seikagaku America, Inc.) was performed. The reaction proceeded for 15 minutes at 37° C., and then at 42° C. for an additional 30 minutes. The reverse transcriptase product, was subject to PCR amplification in the presence of the down stream primer (5′-CCTCCATCCAGGAACATGAG-3′) and a PCR amplification kit (Perkin-Elmer). The product was analyzed on a 1% agarose gel.

For Northern analysis, samples (10 μg total RNA) were heated at 70° C. for 10 minutes in loading buffer (DEPC water, 1×MOPS, 6.6% formaldehyde, 50% formamide, 5% glycerol, bromophenol blue), and then separated by 6.3% formaldehyde-1% agarose gel electrophoresis. RNA was transfered onto Zetaprobe membranes in 10×SSC buffer and hybridized to the random primed 0.7 kb ³²P-bovine SR cDNA probe described above (FIG. 1) at 65° C. Blots were first washed with 0.1×SSC/0.1% SDS at room temperature for 10 minutes, and then at 50° C. for additional 10 minutes. Blots were exposed to X-Omat AR film (Eastman Kodak, Rochester, N.Y.). In a separate northern blot experiment it was shown that the ³²P-bovine SR cDNA probe does not recognize the endogenous mouse SR; similarly, a 0.2 kb ³²P-mouse SR cDNA probe was shown to be specific for the mouse SR. To estimate hepatic bovine SR relative abundance to that of the endogenous mouse SR mRNA, duplicate northern blots of hepatic mRNA from control and TgSR+/− mice were hybridized to either the mouse or bovine specific SR cDNA probe and processed in a similar fashion as above.

Northern blot analysis was also used to quantitate endogenous hepatic 7α-hydroxylase and actin mRNA levels in control and TgSR+/− mice fed a HFHC diet. Total liver mRNA (10 μg/lane) was electrophoresed on a formaldehyde gel and then transfered in 20×SSC buffer to a nitrocellulose membrane (Schleicher & Schuell, Inc. Keene, N.H.). The membrane was baked for 1.5 hours at 80° C., prehybridized, and then hybridized at 62° C. using formamide conditions. Both 0.3 kb mouse 7α-hydroxylase and mouse actin riboprobes were generated using a run off kit (Riboprobe Gemini II Core System, Promega) and ³²P-CTP (Amersham). The membranes were subject to three 10-minute 2×SSC/0.2% SDS washes, first at 40° C., then at 50° C., and then at 62° C. (7α-hydroxylase) or 50° C. (actin). For 7α-hydroxylase two additional washes continued at 65° C. in 0.1×SSC/0.2% SDS for 10 minutes and then for 20 minutes. For actin, one additional wash continued at 50° C. in 0.1×SSC/0.2% SDS for 10 minutes. Image analysis and quantitation of Northern bands were determined on a Molecular Dynamics 400E Phosphoimager (Molecular Dynamics, Sunnyvale, Calif.).

### EXAMPLE 4

Protein Quantification

For different portions of these studies, protein was determined with either the BCA protein assay reagent (PIERCE, Rockford, Ill.), by the method of Bradford (Bradford M. M., Anal. Biochem., 72:248-254 (1976)) or Lowry et al. (Lowry O. H., Rosebrough N. J., Farr A. C., Randall R. J., J. Biol. Chem., 193:265-275 (1951)). In all cases bovine serum albumin was used as a standard.

### EXAMPLE 5

Western Blot Analysis

Liver membranes were isolated from control and TgSR+/− mice according to the method of Via, et al. (Via D. P., Dresel H. A., Gotto A. M. Jr., Methods in Enzymology, 129:216-226 (1986)). Briefly, livers were homogenized in 50 mM Tris-HCl, 150 mM NaCl, 1 mM EDTA, 0.5 mM phenylmethylsulfonyl fluoride (PMSF), and 10 U/mL aprotinin, pH 8.0 (4 mL/g tissue), and spun by at 1500 g for 10 minutes at 4° C. to remove cellular debris. Supernatants were centrifuged at 100,000 g (40,000 rpm in a Beckman Ti60 rotor) for 1 hour at 4° C., and membrane pellets were resuspended in ice-cold 40 mM octyl β-glucopyranoside in 50 mM Tris-HCl, 150 mM NaCl, 1 mM EDTA, 0.5 mM phenylmethylsulfonyl fluoride (PMSF), and 10 U/mL aprotinin, pH 8.0. Nonreduced membrane proteins were electrophoresed on 7.5% SDS polyacrylamide gels and transfered electrophoretically (100 V for 1.5 hours at room temperature) to nitrocellulose membranes. Membranes were blocked with 5% nonfat dried milk (blotto) in 50 mM Tris-HCl, 150 mM NaCl, pH 8.0 and then incubated with guinea pig anti-bovine SR IgG (DeJager S., Mietus-Synder M., Pitas R. E., Arterioscler. Thromb., 13:371-378 (1993); Pitas R. E., Friera A., McGuire J., DeJager S., Arterioscler. Thromb., 12:1235-1244 (1992)). Following incubation with goat anti-rabbit IgG (which cross reacts with guinea pig IgG) conjugated to alkaline phosphatase, the bovine SR-antibody complexes were visualized with an ECL detection system (Amersham).

### EXAMPLE 6

LDL Isolation and Modifications

Human LDL (hLDL) was isolated by sequential ultracentrifugation between the density intervals of 1.019 to 1.050 g/mL (Havel R. J., Eder H. A., Bragdon J. H., J. Clin. Invest., 34:1345-1353 (1955)). hLDL was acetylated with acetic anhydride (ac-hLDL) (Goldstein J. L., Ho Y. K., Basu S. K., Brown M. S., supra., 1979) and used in fluorescence studies (see below). Ac-hLDL was radiolabeled with 125I by the iodine monochloride method of MacFarlane (McFarlane A. S., Nature, 182:53 (1958)) and was used for kinetic studies (see below).

### EXAMPLE 7

Fluorescence Histochemistry

Ac-hLDL was labeled with DiI (1,1′-dioctadecyl-3,3,3′,3′-tetramethylindocarbocyanine perchlorate) according to the method of Voyta, et al. (Voyta J. C., Via D. P., Butterfield C. E., Zetter B. R., J. Cell Biol., 99:2034-2040 (1984)). Control and TgSR+/− mice were tail vein injected with DiI ac-hLDL (320 μg, 1.6 μg/μL). After 10 minutes, mice were sacrificed and liver tissue was rinsed in PBS and cut into pieces for embedding in OCT (Baxter) on dry ice. Cryostat sections (3-5 μm) were placed on polylysine-coated slides and analyzed by fluorescence microscopy using a rhodamine filter set.

### EXAMPLE 8

In Vivo Clearance of Acetylated LDL

The kinetics of ¹²⁵I-ac-hLDL clearance in five TgSR+/− and five control mice was determined. Mice were tail vein injected with ¹²⁵I-ac-hLDL (1.6 mg protein, 0.2 mL). Orbitalsinus blood samples (10 μL) were collected periodically up to 8 minutes in heparinized microcapillary tubes. Radioactivity data are expressed as percent of the first 20-second time point. To control for nonscavenger receptor mediated ¹²⁵I-ac-hLDL clearance, three TgSR+/− and three control mice were coinjected with 0.1 mL of ¹²⁵I-ac-hLDL preparation plus 0.1 mL Fucoidan (10 mg/mL) (Brown M. S., Goldstein J. L., Krieger M., Ho Y. K., Anderson R. G. W., supra., 1979).

### EXAMPLE 9

Food Consumption Studies

Five TgSR+/− and five control mice were maintained on a high-fat, high-cholesterol (HFHC) diet (Diet D12336, Research Diets, Inc., New Brunswick, N.J.) for 3 weeks in individual metabolic cages. The HFHC diet was similar to the atherogenic diet used by Paigen, et al. (Paigen B., Morrow A., Bradon C., Mitchell D., Holmes P., Atherosclerosis, 57:65-73 (1985)), and contained 1.25% cholesterol, 16% fat (5% soy bean oil, 7.5% cocoa butter, and 3.5% coconut oil), and 0.5% cholic acid. The selected animals had an average body weight of 22 to 25 g and were 2 months old. Each group consisted of three males and two females. The weekly amounts of diet consumed by each animal was calculated at Days 7, 14, and 21.

### EXAMPLE 10

Feeding Study

Five TgSR+/−, four TgSR+/+, and five control mice maintained on chow were fasted for 7 to 8 hours prior to obtaining 0.3 mL blood from the tail while under Metofane (Pro-Vet) anesthesia. Mice were then put on the HFHC diet for 3 weeks. Mice were bleed weekly following a 7- to 8-hour fast.

### EXAMPLE 11

Lipoprotein and Lipid Analysis

Lipoprotein total cholesterol distribution in 10 μL plasma samples was determined continuously on-line in the postcolumn eluant following Superose 6 (Pharmacia Biotech Inc., Piscataway, N.J.) high performance gel-filtration chromatography essentially as described (Kieft K. A., Bocan T. M. A., Krause B. R., J. Lipid Res., 32:859-866 (1991); Aalto-Setälä K., Bisgaier C. L., Ho A., et al., J. Clin. Invest., 93:1776-1786 (1994)) except that we used a Rainin HPLC and Dynamax Compare software (Rainin Instrument Co., Inc., Woburn, Mass.) for instrumentation and data reduction, respectively. Total plasma triglycerides were determined enzymatically with a commercially available kit (Trigli-cinet 2 kit, Sclavo Inc., Wayne, N.J.). Total plasma cholesterols were determined enzymatically according to the method of Allain, et al. (Allain C. C., Poon L. S., Chan C. S. G., Richmond W., Fu P. C., Clin. Chem., 20:470-475 (1974)).

### EXAMPLE 12

Analysis of Hepatic Lipids

Major hepatic lipid classes were determined in five TgSR+/− and four control mice that were on the HFHC diet for 25 days. Livers (0.5 g) were homogenized in a total volume of 5 mL phosphate-buffered saline. Aliquots were removed for protein determination (Lowry O. H., Rosebrough N. J., Farr A. C., Randall R. J., supra., 1951) and extraction of liver lipids. Homogenized liver (1.0 mL) was extracted with 6 mL ethyl acetate/acetone (2/1:v/v) containing 0.01% butylated hydroxytoluene and a 4-hydroxy-cholesterol (1 mg) internal standard in teflon-lined screw-cap 20-mL glass tubes according to the method of Slayback, et al. (Slayback J. R. B., Cheung L. W. Y., Geyer R. P., Anal. Biochem., 83:372-384 (1977)). Samples were vigorously mixed for 10 minutes and extraction continued overnight. Following addition of 2 mL water, and 5 minutes low speed centrifugation (500 rpm), the upper phase containing both polar and nonpolar lipids was removed and evaporated to dryness under nitrogen. Residual solvent was removed by lyophilization. Dried lipids were solubilized in 200 μL of iso-octane/tetrahydrofuran (97/3:v/v) and 5 μL were injected onto a 4.6×100 mM silica column equibrated with iso-octane/tetrahydrofuran (97/3:v/v) on a Spectra Physics HPLC by a modification of the method of Christie (Christie W. W., J. Lipid Res., 26:507-512 (1985)). Postcolumn eluant was detected in a evaporative light scattering detector (Varex, Model ELSD IIA). Authentic lipid standards were utilized to calibrate the detector response for the various major lipid classes.

### EXAMPLE 13

Determination of Fecal Bile Acids

Five TgSR+/− and five control mice were maintained on chow diets in individual metabolic cages for 1 week, followed by a high-fat, high-cholesterol diet for 3 weeks. Total feces from each mouse was collected at the end of each week and stored at −20° C. Total fecal bile acids was determined by the fluorescence method of Beher, et al. (Beher W. T., Strandnieks S., Lin G. J., Sanfield J., Steroids, 38:281-295 (1981)). Briefly, feces was homogenized in three volumes of water. An aliquot of the fecal homogenate (1 g) was mixed with 7 mL of ethanol and heated to 70° C. for 30 minutes. The mixture was then filtered through a pleated filter and washed once with 6 mL of preheated ethanol. A 4-mL aliquot from each sample was dried under nitrogen and then dissolved in 2 mL of 3 M NaOH and heated at 100° C. for 2 hours. Samples (10 μL), 2.4 mL of tris buffer pH 9 and 0.5 mL of reagent (2 mg resazurin, 100 mg β-NAD, 6.4 units of hydroxysteroid oxidoreductase and 37 units of diaphorase in 100 mL of 0.05 M pH 7.4 phosphate buffer containing 19.1 mg sucrose, 0.1 μg dithioerythritol, 7.5 mg EDTA, and 50 mg bovine serum albumin) were incubated at room temperature for 1.5 hours. Samples were excitated at 565 nm and emission fluorescence determined at 580 nM in a fluorescence spectrophotometer model LS-3 (Perkin-Elmer, Oakbrook, Ill.). Standards of cholic acid were used to calibrate the assay.

### EXAMPLE 14

Cholesterol Absorption

Cholesterol absorption was determined in three control and five TgSR+/− mice by determination of the differential absorption of cholesterol and β-sitosterol on a HFHC diet. Briefly, mice individually housed in metabolic cages were maintained ad libitum on a chow diet prior to intragastric bolus administration of ³H-cholesterol (1.5 μCi) plus ¹⁴C-β-sitosterol (0.1 μCi) in 100 μL sunflower seed oil. Mice were then allowed ad libitum access to the HFHC diet for 4 days. An aliquot of the oral dose and a homogenate of the feces collected over the 4 days were extracted with ethyl acetate/acetone (2/1:v/v) and processed in a similar fashion as described above for extraction of hepatic lipids. Radioactivity in an aliquot of the lipid phase was determined by liquid scintillation counting. The ratio of ³H to ¹⁴C in the extracts were determined and used to estimate percent cholesterol absorption by the following formula:

$$\begin{matrix}Percent Cholesterol \\ Absorption\end{matrix} = 100 \times \frac{\begin{matrix}( {(}^{3} H {/}^{14} C in Oral Dose ) - \\ ( {(}^{3} H {/}^{14} C in Feces ) )\end{matrix}}{{(}^{3} H {/}^{14} C in Oral Dose )}$$

1. A method for introducing a scavenger receptor gene into the liver of a mammal to make said mammal resistant to atherosclerosis, comprising introducing the DNA into a mammal by a process of delivery selected from the group consisting of:
(a) use of calcium phosphate coprecipitation;
(b) in a complex of cationic liposomes;
(c) electroporation;
(d) receptor-mediated endocytosis;
(e) naked DNA;
(f) transduction by a viral vector;
(g) particle-mediated gene transfer; and
(h) synthetic peptides.

2. The method of claim 1 wherein the mammal is a human.

3. A method for introducing a scavenger receptor gene into a mammal to make said mammal resistant to atherosclerosis comprising inserting said scavenger receptor gene into a vector and expressing the scavenger receptor in the liver of said mammal.

4. The method of claim 3 wherein the mammal is a human.

5. The ectopic expression of a scavenger receptor in the liver of a mammal for the reduction of apo B containing lipoproteins, elevation of high-density lipoprotein cholesterol, and prevention of atherosclerosis.

6. The ectopic expression of a scavenger receptor according to claim 5 wherein the mammal is a human.

7. The ectopic expression of a scavenger receptor according to claim 5 wherein expression is transient expression in the liver.

8. The ectopic expression of a scavenger receptor according to claim 5 wherein expression is stable expression in the liver.

9. A method of treating atherosclerosis; hyperbetalipoproteinemia; hypercholesterolemia; hypertriglyceridemia; hypoalphalipoproteinemia; vascular complications of diabetes; transplant, atherectomy, and angioplastic restenosis in a patient comprising administering to the liver of said patient a therapeutically effective amount of a scavenger receptor gene.

10. A method of treating atherosclerosis; hyperbetalipoproteinemia; hypercholesterolemia; hypertriglyceridemia; hypoalphalipoproteinemia; vascular complications of diabetes; transplant, atherectomy, and angioplastic restenosis in a patient comprising administering to the liver of said patient a therapeutically effective amount of a scavenger receptor gene in combination with one or more agents selected from the group consisting of:
(a) ACAT inhibitor;
(b) HMG-CoA reductase inhibitor;
(c) lipid regulator; and
(d) bile acid sequestrant.

11. A pharmaceutical delivery method adapted for hepatic administration to a patient in an effective amount of an agent for treating atherosclerosis; hyperbetalipoproteinemia; hypertriglyceridemia; hypercholesterolemia; hypoalphalipoproteinemia; vascular complications of diabetes; transplant, atherectomy, and angioplastic restenosis comprising a scavenger receptor gene and a suitable viral or nonviral delivery system.

12. A pharmaceutical delivery method according to claim 11 adapted for ex vivo or in vivo delivery.

13. A pharmaceutical delivery method according to claim 11 directed to therapeutic or prophylactic administration.
